(19) United States
(12) Patent Application Publication
(10) Pub. No.: US 2021/0236131 A1
(43) Pub. Date: Aug. 5, 2021

(54) BIODEGRADABLE INTRALUMINAL SMALL INTESTINAL ANASTOMOTIC GUIDE

(71) Applicants: University of Tennessee Research Foundation, Knoxville, TN (US); The Board Of Trustees Of The University Of Arkansas, Little Rock, AR (US)

(72) Inventors: David Edgar Anderson, Knoxville, TN (US); Alexandru Biris, Knoxville, TN (US); Karrer M. Alghazali, Knoxville, TN (US); Alisha Potter Pedersen, Knoxville, TN (US)

(21) Appl. No.: 17/259,802

(22) PCT Filed: Jul. 12, 2019
(86) PCT No.: PCT/US2019/041550

§ 371 Date: Jan. 12, 2021

Related U.S. Application Data
(60) Provisional application No. 62/697,475, filed on Jul. 13, 2018.

(51) Int. Cl. A61B 17/11; A61L 31/10; A61L 31/16; B32B 1/08; B32B 7/12; B32B 27/08

(52) U.S. Cl. CPC A61B 17/1114; A61L 31/10; A61L 31/16; A61B 2017/00004; B32B 7/12; B32B 27/08; A61B 2017/1132; B32B 1/08

(57) ABSTRACT

The present invention is directed to guides for use in anastomotic procedures, and in particular in small intestine anastomosis. An anastomotic guide of the invention preferably comprises a tubular body comprising a wall having an abluminal surface, a luminal surface and two ends; the wall comprising at least one sheet of a biocompatible material in a laminate structure having two or more layers, the layers of the laminate structure being joined by a water soluble adhesive polymer, the tubular body being insertable into the lumen of an organ having a luminal surface so that the abluminal surface of the anastomotic guide contacts the luminal surface of the organ.

This application claims priority to U.S. Provisional Application No. 62/697,475, filed Jul. 13, 2018, the entire contents of which are incorporated herein by reference.

### FIELD OF THE INVENTION

The present invention is directed to guides for use in anastomotic procedures, and in particular in small intestine anastomosis.

### BACKGROUND OF THE INVENTION

Intestinal anastomosis is a common procedure performed in both emergency and elective situations. Anastomosis restores continuity to the bowel following resection and can allow bypass of unresectable bowel.[1] Numerous pathologic conditions indicate the need for intestinal anastomosis, including vascular compromise, bowel gangrene, obstruction, intussusception, volvulus, polyps, neoplasia, ascarid impaction, perforation due to trauma, severe inflammatory bowel disease refractory to medical therapy, chronic constipation, various congenital abnormalities, severe inflammation due to disease, etc.[1] These indications exist in both human and veterinary medicine alike.

Several devices have been described for use in anastomotic procedures. U.S. Pat. No. 5,180,392 discloses a prosthesis for joining tubular organs that comprises a fragmentable body that can be crushed following anastomosis. U.S. Pat. No. 9,974,543 discloses an anastomotic connector that includes a biocompatible liner that is not degradable surrounded by a bioabsorbable shell. U.S. Pat. No. 9,820,746 discloses an expandable tissue scaffold for use at an anastomotic site.

Numerous risks are associated with intestinal anastomosis, including stricture which leads to intestinal obstruction, leakage at the site which can lead to peritonitis, sepsis or abscessation, poor vascularization leading to necrosis, formation of adhesions, and the need for prolonged anesthesia to complete the surgery. What is needed in the art is an anastomotic guide that allows lowers the risk of obstruction, peritonitis, sepsis, necrosis and adhesions and reduces the time needed to complete anastomosis.

### SUMMARY OF THE INVENTION

The present invention is directed to guides for use in anastomotic procedures, and in particular in small intestine anastomosis.

In some preferred embodiments, the present invention provides anastomotic guides comprising: a tubular or cylindrical body comprising a wall having an abluminal surface, a luminal surface and two ends; the ends can be of a smaller or larger diameter compared to the body of the device; the wall comprising at least one sheet of a biocompatible material in a laminate structure having one, two or more layers, the layers of the laminate structure being joined by a water soluble adhesive polymer, the tubular body being insertable into the lumen of an organ having a luminal surface so that the abluminal surface of the anastomotic guide contacts the luminal surface of the organ. The body can also be composed of a cylindrical structure that has non regular porosities of a variety of dimensions, ranging from 1 nm to several centimeters, formed within its structure. The structure could be made of one or multiple biocompatible and biodegradable polymer(s) or materials.

In some preferred embodiments, the biocompatible material is selected from the group consisting of (Poly(α-esters), Polyglycolide, Polylactide, Poly(L-lactic acid) (PLLA), Poly(D-lactic acid) (PDLA), Poly(D,L-lactic acid) (PDLLA), Poly(lactide-co-glycolide), Polyhydroxyalkanoates, Poly(3-hydroxybutyrate), Poly(3-hydroxybutyrate-co-3-hydroxyvalerate (PHBV), Polycaprolactone (PCL), Poly(propylene fumarate) (PPF), Polyanhydrides, Polyacetals, Poly(ortho esters), Polycarbonates, Poly(trimethylene carbonate) (PTMC), Poly(desaminotyrosyltyrosine alkyl ester carbonates) (PDTEs), Polyurethanes, Polyphosphazenes, (Poly[bis(trifluoroethoxy)phosphazene], Polyphosphoesters, Poly(ester ether)s, Polydioxanone (PDO), Poly(β-amino esters) (PBAEs), Poly(anhydride ester)s, Poly(ester urethane)s, Poly(ethylene glycol) (PEG), Poly(propylene glycol) (PPG), triblock pluronic ([PEG]n-[PPG]m-[PEG]n), pluronic, PEG diacrylate (PEGDA), PEG dimethacrylate (PEGDMA), collagen, (Collagen types I, II, III, and IV), elastin and elastin-like polypeptides (ELPs), albumin, fibrin, natural poly(amino acids), poly(γ-glutamic acid), poly(L-lysine), synthetic poly(amino acids), poly(L-glutamic acid), poly(aspartic acid), poly(aspartic acid) (PAA), polysaccharides, hyaluronic acid (HA), chondroitin sulfate (CS), chitin, chitosan, alginate, dextran, agarose, mannan and inulin and combinations thereof. In some preferred embodiments, the biocompatible material is a biodegradable material. In some preferred embodiments, the biodegradable material is water soluble. In some preferred embodiments, the sheet of biocompatible material is porous.

In some preferred embodiments, the water soluble adhesive polymer is selected from the group consisting of Polyvinyl alcohol, Poly(ethylene glycol), Polyvinyl pyrrolidon, Polyacrylic acid (PAA), Polyacrylamides, N-(2-Hydroxypropyl) methacrylamide (HPMA), Polyoxazoline, Polyphosphates, Polyphosphazenes, xanthan gum, pectins, carrageenan, Cellulose ethers, Carboxymethyl cellulose (CMC), (Hydroxypropyl Cellulose (HPC), Hydroxypropylmethyl Cellulose (HPMC), hyaluronic acid (HA), albumin, starch, starch-based derivatives, and combinations thereof. In some preferred embodiments, the water soluble adhesive polymer has a dissolution rate in water or aqueous solution that is greater than the dissolution rate of the at least one sheet of biocompatible material.

In some preferred embodiments, the guide further comprises a plurality of sheets of biocompatible material or mixtures of multiple such materials in a laminate or cylindrical structure having one, two or more layers, the layers of the laminate structure being joined by the water soluble adhesive polymer.

In some preferred embodiments, the tubular body has a diameter compatible (smaller, identical or larger) with insertion into the small intestine of a mammal so that the abluminal surface of the tubular body engages the luminal surface of the small intestine so that the guide is maintained in place at the site of anastomosis.

In some preferred embodiments, the mammal is selected from the group consisting of humans, or animals such as but not limited to non-human primates, pigs, horses, cows, sheep, goats, camelids, dogs and cats.

In some preferred embodiments, the guides further comprise one or more retention members positioned proximal to one or both of the two ends of the tubular body, the retention member(s) providing pressure to the luminal surface of the organ so that when the anastomotic guide is inserted into the organ the position of the anastomotic guide is maintained relative to the luminal surface of the organ. In some preferred embodiments, the tubular body has a center and has one or more grooves distal to the center of the tubular body that extend around the tubular body so that when the anastomotic guide is inserted into an organ, the grooves are engageable by a retention member external to the organ to maintain the position of the anastomotic guide relative to the luminal surface of the organ.

In some preferred embodiments, one or both of the biocompatible polymer(s) and water soluble adhesive polymer comprise a therapeutic agent. In some preferred embodiments, the therapeutic agent is an antimicrobial agent.

In some preferred embodiments, the present invention provides anastomotic guides comprising: a tubular body comprising a wall having an abluminal surface, a luminal surface and two ends; the wall comprising at least one sheet of a porous biodegradable material in a laminate structure having one, two or more layers, the layers of the laminate structure being possibly joined by a water soluble adhesive polymer so that the tubular body is insertable into the lumen of an organ having a luminal surface so that the abluminal surface of the anastomotic guide contacts the luminal surface of the organ, wherein the tubular body has a diameter compatible with insertion into the small intestine of a mammal selected from the group consisting of humans, or animals such as but not limited to non-human primates, pigs, horses, cows, sheep, goats, camelids, dogs and cats.

In some preferred embodiments, the present invention provides anastomosis procedures comprising: inserting an anastomotic guide as described above into first and second ends of an organ having a lumen so that the anastomotic guide extends between the first and second ends, and joining the first and second ends by anastomosis. In some preferred embodiments, the anastomosis comprises joining the first and second ends of the organ by sutures. In some preferred embodiments, the anastomosis comprises joining the first and second ends of the organ by staples. In some preferred embodiments, the anastomosis joins ends of an organ resulting from resection of the organ. In some preferred embodiments, the organ is the small intestine of a mammal. In some preferred embodiments, the mammal is selected from the group consisting of humans, animals such as but not limited to non-human primates, pigs, horses, cows, sheep, goats, camelids, dogs and cats. In some preferred embodiments, the anastomotic guide degrades, softens or loses its structural integrity within a period of from 1 minute to 90 days.

In some preferred embodiments, the present invention provides for use of an anastomotic guide as described above to join two ends of an organ in a subject requiring anastomosis.

### BRIEF DESCRIPTION OF THE DRAWINGS

FIG. 1 is a perspective view of one embodiment of an anastomotic guide of the present invention.

FIG. 2. is a side view of one embodiment of an anastomotic guide of the present invention.

FIG. 3 is a side view of another embodiment of an anastomotic guide of the present invention.

FIGS. 4A and 4B provide a schematic diagram of one method of making the anastomotic guides of the present invention.

FIGS. 5A and 5B are schematic diagrams depicting alternative arrangements of biocompatible polymer sheets that can be used during fabrication of anastomotic guides of the present invention.

### DETAILED DESCRIPTION OF THE INVENTION

The present invention is directed to guides for use in anastomotic procedures, and in particular in small intestine anastomosis. As will be described in more detail below, in some preferred embodiments, the anastomotic guides of the present invention comprise a tubular or cylindrical body comprising a wall having an abluminal surface, a luminal surface and two ends. In still further preferred embodiments, the wall comprises at least one sheet of a biocompatible material in a laminate structure having two or more layers, the layers of the laminate structure being joined by a water soluble adhesive polymer so that the tubular body is insertable into the lumen of an organ having a luminal surface so that the abluminal surface of the anastomotic guide contacts the luminal surface of the organ. The anastomotic guides of the present invention provide for a decrease in adverse events associated with anastomosis such as obstruction, peritonitis, sepsis, necrosis and adhesions, and further decreases the time needed for anastomosis which reduces complications arising from prolonged anesthesia.

There are various factors necessary to consider prior to performance of an intestinal anastomosis. High risk conditions may contraindicate the performance of the procedure, particularly in cases of elective resection, even if the underlying condition warrants an intestinal anastomosis. Such high risk conditions include severe sepsis, peritonitis, significant hypoalbuminemia, systemic illness, unlikely viability of bowel, etc.[1] Pre-operative medical therapy should be instituted in these cases to stabilize the patient in preparation for surgical correction of the underlying condition. There are also many individual patient factors that can contribute to altered healing of the anastomotic site.[3]

Once surgery is elected or required, perioperative management of the patient includes fluid administration and antibiotic prophylaxis. Nasogastric tube placement, urinary catheter placement, and venous thromboembolism prophylaxis are also commonly instituted in human patients.[1]

Practices that are essential for the best post-operative recovery potential of the patient, regardless of the specific anastomotic techniques employed, include adequate accessibility of the bowel segment affected, gentle manipulation of the bowel and surrounding abdominal structures, appropriate hemostasis and maintenance of vascularization following transection, avoidance of tension at the anastomotic site, proper surgical technique, and avoidance of contamination of the abdomen with intestinal contents.[1]

There are numerous options for operative techniques that can be utilized in the performance of a small intestinal anastomosis. Surgeons select from these techniques based on the particular situation, personal preference, data demonstrating benefits or hindrances of utilizing specific techniques, cost, feasibility, availability of instruments, the diameter of the affected area of bowel, presence or lack of edema, location within the abdominal cavity, type of disease or condition, and time constraints.[1,3] An exploratory laparotomy may be performed [2] to ascertain the condition of the patient, which can aid in further planning of the procedure

In most operations, a midline incision is performed to gain access to the small intestine, although a supraumbilical transverse incision is often used in small children. Self-retaining retractors maintain exposure to the abdominal cavity. The diseased segment of bowel is mobilized and exteriorized to allow for resection and to decrease tension at the anastomotic site. The mesentery is also transected to aid with this, taking care to ligate vessels while still maintaining the vascular arcade supplying the bowel to be anastomosed. Noncrushing clamps are placed on the bowel to be maintained, and crushing clamps are placed on the segment to be resected, both on the antimesenteric side. This prevents spillage of intestinal contents into the abdominal cavity of the patient upon transection. When using a hand-sewn method, an oblique transection of the bowel is made close to each crushing clamp and the diseased bowel is removed. [2]

There are many techniques that can be opted for in the performance of a small intestinal anastomosis. Categories of hand-sewn anastomoses include: simple continuous suture pattern versus interrupted suture pattern; single-layered or double-layered; end-to-end or side-to-side; use of absorbable versus nonabsorbable suture material (and choice of a specific type amongst those categories); extramucosal or full-thickness suture bites; and choice of variable spacing between bites. Categories of stapled anastomoses include: end-to-end or side-to-side; oversewing the stapled area, burying it, or no additional modifications; and variable choice of stapling device employed.[3]

In a double-layered hand-sewn enteroenterostomy, as is commonly performed, the two cut ends are opposed and stay sutures of 3-0 silk are introduced between the serosa roughly 5-mm from the cut edges. Interrupted sutures of 3-0 silk in a Lembert pattern are placed within the seromuscular layer between the stay sutures to form the posterior outer layer, with 3-mm being spaced between each interrupted suture. A single Connell suture is placed between the two cut ends. The posterior and anterior inner layers are formed from full-thickness interrupted sutures starting from the near and far end, respectively. 3-0 polyglactin is utilized for these layers and knots are placed inside the lumen of the bowel. To avoid extrusion of mucosa, a large portion of the seromuscular layer and a small portion of mucosa is taken in each bite. Interrupted Lembert sutures are placed to form the anterior outer seromusculuar layer. Lumen patency is ensured by palpation and the mesenteric defect is closed with interrupted sutures of 3-0 silk.[2] Stapling is an alternative method of performing an anastomosis. In an anatomic end-to-end stapled anastomosis, three traction sutures are placed in the cut ends of the bowel in a triangle-shape and a noncutting linear stapler is fired between each of the sutures. Excess tissue is then removed, with the final result being an everted anastomosis. More commonly, a stapled end-to-end anastomosis is performed in a functional manner. The cut ends of bowel are opposed and the two forks of a linear cutting stapler are placed either into the lumens of the cut ends or through enterotomies made in the antimesenteric border of the two segments after the cut edges have been stapled closed. The stapler is fired and forms a lumen from the walls between the segments. The cut ends or enterotomies are closed with staples or sutures. If bleeding occurs from the stapled site, underrunning sutures are placed.[2]

In children, small intestinal anastomoses are generally performed in a single layer of interrupted sutures composed of polyglactin; however, intraluminal staplers have also been used. [2]

Regardless of the technique utilized, there are several complications that frequently occur during or following an intestinal anastomosis procedure. A complication that may present itself early in the recovery period is leakage from the anastomotic site. During the first 5-7 days of the recovery, the efficacy of the anastomotic site largely relies on the holding ability of the suture material or staples. Should a leakage occur within the first day or two postoperatively, it is most likely due to the techniques utilized to perform the anastomosis. If leakage occurs around one week postoperatively, it is likely due to negative effects from normal healing. Leakage may take the form of diffuse peritonitis or localized abscessation, the former having a high morbidity and mortality rate and requiring additional surgical intervention.[2] Leakage increases the mortality rate of bowel anastomosis from 7.2% to 22%.[3,4]

Another commonly encountered complication is bleeding, either intraoperatively or postoperatively. Evidence of intraoperative bleeding at the anastomotic site not only is evidenced by blood exuding into the abdomen, but can include viewing blood within the lumen distal to the anastomosis. The integrity of the anastomosis should be reevaluated if this occurs and hemostatic sutures placed if necessary. Postoperative bleeding is evident as hematemesis, melena, bleeding from an intraabdominal drain, etc. These cases should be treated with medical management or, if severe, surgical intervention. Stapled anastomoses in particular have been shown to result in disruption of mesenteric blood vessels, resulting in ischemia. [2] Incision site infections may occur following an open abdominal procedure such as intestinal anastomosis. This is often due to contamination from intestinal contents during the procedure. A drain can be placed to manage the infection. Anastomotic stricture is also a serious late complication with a slightly higher prevalence following a stapled end-to-end anastomosis. The most important risk factor contributing to the development of a stricture postoperatively is treatment of a controlled anastomotic leak with conservative medical management. Dilatation or surgical revision may be necessary to treat this complication. [2]

There are many controversies regarding the most beneficial techniques for an intestinal anastomosis procedure, including suturing or stapling, type of suture material selected, single- or double-layered suturing, continuous or interrupted suture pattern, and inverting or everting the anastomosis.

Commonly used varieties of suture material that experience minimal fraying and remain strong for the duration necessary include absorbable polyglactin, polyglycolic acid, and chromic catgut. Polyglactin and polyglycolic acid also result in minimal inflammation. Silk is commonly used should a nonabsorbable variety be preferred, although it incites a more pronounced inflammatory reaction. [2,5] Polypropylene is also nonabsorbable and incites less inflammation. For an anastomosis with two layers, generally the inner layer is formed with absorbable sutures and the outer layer with silk. For a single layer, silk is typically employed.[2]

Most often intestinal anastomoses are performed in a double-layer, however this takes more time and is somewhat more difficult of a procedure. Performing a single-layered anastomosis can reduce the time required to perform the procedure, which also reduces the cost. Many trials and meta-analyses have also demonstrated that there is no increased risk of leakage, perioperative complications, mortality, or hospital recovery time when a single-layered anastomosis is performed as opposed to a double-layered. [2,6-9] Utilizing a simple continuous suture pattern can greatly reduce the time it takes to perform an intestinal anastomosis, as well as produce a more tight seal between the bites and improve hemostasis. A downfall of this is that a disruption in the suture may result in an increased likelihood of dehiscence than if simple interrupted sutures are employed. Studies have demonstrated that blood flow and oxygenation at the anastomotic site is decreased with simple continuous suturing;[2,10] however, trials have not demonstrated differences in complication rates when compared to simple interrupted suturing.[2,6]

Utilizing an inverting anastomosis technique is currently the most common practice. A study conducted in 1966 determined that inverted anastomoses were weaker than everting anastomoses, [2,11] however a later study determined that, in colorectal anastomoses, fecal fistulas are more likely to occur in everting than inverting anastomoses.[2,12] Stapled anastomoses may be increasing in prevalence due to the increase in availability of stapling devices, yet studies have variable results when comparing the incidence of anastomotic leakage and other complications between stapled and sutured anastomoses. Previously, several case series, small randomized controlled trials, and a meta-analysis on anastomoses throughout the gastrointestinal system did not demonstrate a difference in leakage rates, morbidity, or mortality between stapled and sutured anastomoses.[2,3,13-18] Alternatively, two meta-analyses found that there was a higher occurrence of stricture and intraoperative issues in colorectal anastomoses when a stapling technique was utilized[2,19] but a decreased occurrence of leaks in stapled ileocolic anastomoses.[2,20] More recently, studies have examined the use of certain techniques in specific situations to detect any recognizable differences in complication rates. For example, a retrospective study found that the rate of leakage was significantly higher in stapled anastomoses when the procedure was performed as a result of trauma.[3,21] A different study found contradictory results, that being that there was no difference between stapled and sutured anastomosis leakage rates in trauma cases. It did, however, find that enterotomies not requiring resection had better results when sutured.[3,22] A retrospective study examining emergency small and large intestinal anastomoses found that there was increased leakage and intra-abdominal abscess rates in the stapled group.[3,23] While older studies have had conflicting results, a more recent large retrospective evaluation of anastomoses following elective reversal of loop ileostomy and a review of ileocolic anastomoses demonstrated higher leakage rates in sutured anastomoses.[2,3,20,24,25]

While the data on sutured versus stapled methods seems quite variable, studies have demonstrated that sutured anastomoses heal via primary intention and stapled anastomoses heal via secondary intention, which is the likely cause of increased stricture that has been noted with staples. Ultimately, the technique chosen should be based on the particular situation at hand and the preference and experience of the surgeon. One benefit of utilizing a stapling technique is that it is generally quicker and often easier to perform, particularly if the anastomosis is performed in the pelvic region.[1,2] This time reduction may be assumed to be beneficial in cases of emergencies, however it has been shown that this did not ultimately have an effect on patient outcome. [4,27,28]

The considerations and techniques regarding intestinal anastomoses in human medicine are for the most part directly applicable to veterinary medicine. There are some unique concerns and practices found in the literature, however. The most common reasons for resection and anastomosis in canine and feline patients are foreign body entrapment, neoplasia, and bowel damage due to trauma.[29-31]

Suturing is performed far more often than stapling due to familiarity with the process and cost, which is of particular concern in the veterinary field. Multifilament sutures are not recommended due to the dragging they impart on the tissues and the increased likelihood that they will incite an inflammatory reaction.[29,32] Either absorbable or nonabsorbable suture material can be utilized, however nonabsorbable suture should not be used when performing a continuous suture pattern.[29,32,33] When an anastomosis is being performed by a suturing method, it is best to utilize an appositional pattern that avoids both eversion and inversion, as eversion introduces an increased risk of adhesion development, and inversion decreases the size of the lumen.[29,32,34,35] Single-layered suturing is also recommended because performing a double-layered anastomosis may result in a compromised lumen, poor apposition, necrosis, and increased healing time.[29,32] Performing a simple continuous suture pattern reduces the time required for the procedure, allows for better approximation, and results in decreased misalignment of the cut edges of bowel. [29,31,32,36,37] As well, leakage has been noted to occur in 11% of animals with an anastomosis completed with an interrupted suture pattern, versus only 3% with a continuous suture pattern.[29-31,38] Bites are suggested to be 3-mm in width and 3-mm apart from one another; however, this may vary based on the size of the intestines.[29,32] Regardless of technique employed, everted mucosa can be trimmed away or prevented by using a modified Gambee pattern.[29,31]

Electing to perform an anastomosis with staples can reduce the time required to perform the procedure, may reduce manipulation of the bowel, and may increase immediate post-operative burst strength. Stapled anastomoses present a complication rate of 13-14% and result in similar integrity, bursting strength, stenosis, and healing when compared to anastomoses performed with simple interrupted sutures.[29,37,39,40] Utilizing a stapling device presents an added expense, however, as well as additional training; hence, this method is less frequently employed. Regardless of whether the anastomosis is completed by a suturing or stapling method, an additional tactic recommended is to omentalize the anastomosis or perform a serosal patch graft. Both of these additions reduce risks such as leakage and vascular compromise.[29,41] The integrity of the anastomotic site can be tested immediately after being performed by injecting saline into the lumen of the bowel segment and observing for any leaks. If leakage occurs, an interrupted suture should be placed to correct the defect.[29]

The present invention provides devices and methods for improving outcomes for anastomotic procedures. The devices of the present invention are not limited to use in particular organ. In some preferred embodiments, the devices of the present invention find use in anastomotic procedures in any tubular organ with a lumen where contents within the organ are normally discharged from the body. Examples of such organs include, but are not limited to, the esophagus, small intestine, large intestine, rectum, bile dust, pancreatic duct, ureter, urethra, nasolacrimal duct, and vas deferens. In some particularly preferred embodiments, the devices find use in anastomosis of the small intestine.

Accordingly, the present invention provides an anastomotic guide that when inserted into the lumen of a target organ during an anastomotic procedure can maintain its shape, architecture and dimensions for a period of time between 1 sec and 10 years, preferably from about 1 to 5 minutes to about 30 to 180 days, and more preferably from about 1 day to 90 days, and most preferably from about 30 minutes to 3 days, after which the guide will collapse, loose its structural integrity, disintegrate, and/or degrade so that it can be eliminated from the body or totally degraded and absorbed.

In some preferred embodiments, the device is a hollow cylindrical tube comprising layers or films of biocompatible polymer (i.e., support layers) joined in a laminate moisture/fluid degradable polymer (i.e., adhesive layer). In still further preferred embodiments, the device is formed in different shapes and dimensions based on desired use, with diameter ranging, for example, from 1 nm to 30 cm, most preferably from about 1 mm to 10 cm, and lengths ranging from 10 nm to 1 m. In some preferred embodiments, the overall thickness of the wall can vary between 1 nm to 10 cm. In a separate embodiment, the guide is manufactured by a combination of one or multiple polymers that are biodegradable (with identical or dissimilar degradation rates) and porosities ranging from 1 nm to several centimeters, preferably 2-5 cm. The porosities can be interconnected or not. The cylindrically shaped guide could have one or multiple openings or lumens from one end to the other end of the device.

In preferred embodiments, the anastomotic guide of the present invention is insertable into the lumen of an organ having a luminal surface so that the abluminal surface of the anastomotic guide contacts the luminal surface of the organ. In some particularly preferred embodiments, the tubular body has a diameter compatible with insertion into the small intestine of a mammal so that the abluminal surface of the tubular body engages the luminal surface of the small intestine so that the guide is maintained in place at the site of anastomosis.

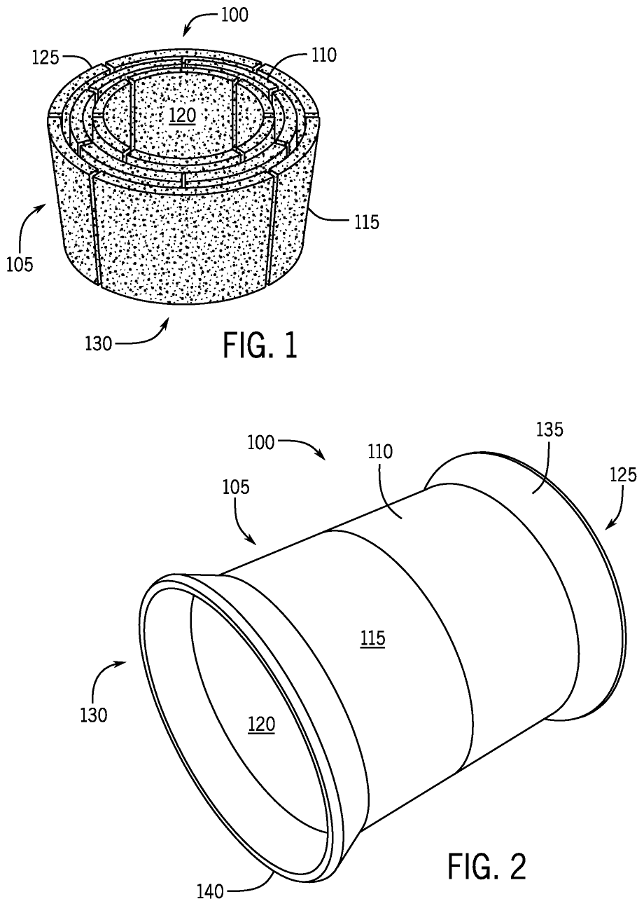

Preferred anastomotic guides are depicted in FIGS. 1, 2 and 3.

Referring to FIG. 1, in some preferred embodiments an anastomotic guide 100 according to the present invention comprises a hollow tubular body 105 comprising a wall 110 having an abluminal surface 115, a luminal surface 120 and two ends 125 and 130. In some preferred embodiments, the wall 110 is formed from a plurality of porous polymer sheets 135 arranged in an overlapping laminate structure that are joined together by a water soluble adhesive polymer.

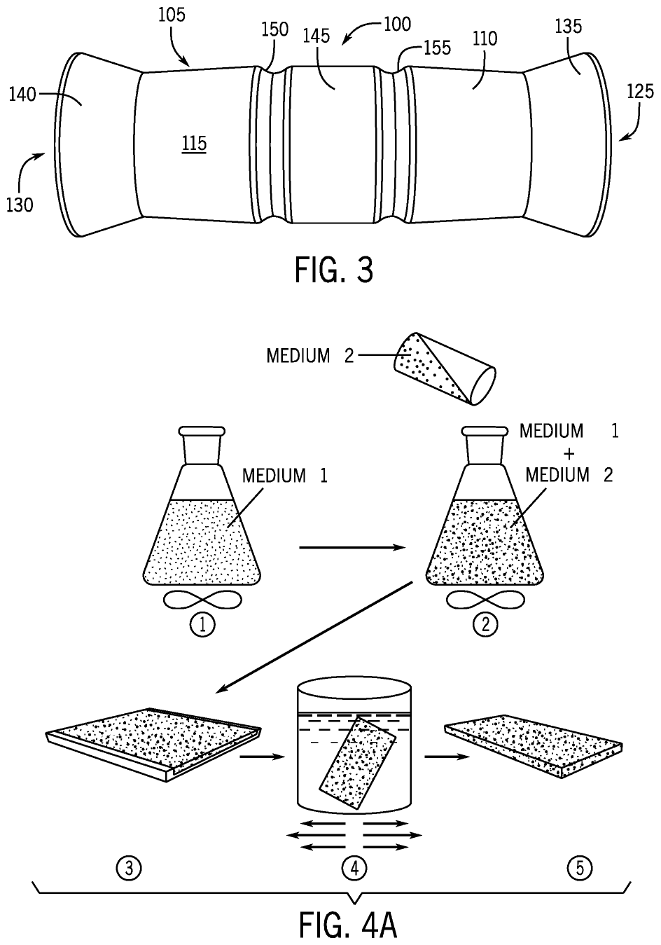

Referring to FIG. 2, in some preferred embodiments an anastomotic guide 100 according to the present invention comprises a hollow tubular body 105 comprising a wall 110 having an abluminal surface 115, a luminal surface 120 and two ends 125 and 130. In some embodiments, the anastomotic guide comprises one or more retention members 135 and 140 positioned proximal to one or both of the two ends of the tubular body. In the depicted embodiments, the retention members are flanges that project outward from the ends of the tubular body. The retention member(s) preferably provide pressure to the luminal surface of an organ so that when the anastomotic guide is inserted into the lumen of the organ the position of the anastomotic guide is maintained relative to the luminal surface of the organ. Referring to FIG. 3, in some embodiments the tubular body 105 has a center portion 145 and has one or more grooves 150 and 155 distal to the center portion 145 of the tubular body that extend around the tubular body so that when the anastomotic guide is inserted into an organ, the grooves are engageable by a retention member (not shown) external to the organ to maintain the position of the anastomotic guide relative to the luminal surface of the organ. In some embodiments, the retention member may be a lock ring (preferably made of a polymer) that is sized to engage the grooves 150 and 155.

In some embodiments, the wall forming the tubular body is made of a biocompatible material. In some preferred embodiments, the biocompatible material is a biodegradable material. In some further preferred embodiments, the biodegradable material is water soluble. In some still further preferred embodiments, the biocompatible material is porous. In some particularly preferred embodiments, the wall comprises at least one sheet of a biocompatible material in a laminate structure having two or more layers, the layers of the laminate structure being joined by a water soluble adhesive polymer. In some preferred embodiments, the tubular body is flexible. In some particularly preferred embodiments, the water soluble adhesive polymer used to join the sheets together has a dissolution rate in water or aqueous solution that is greater or faster than the biocompatible material (which may also be dissolvable in aqueous solution) used in the sheets. Thus, following anastomosis where the guide is used, the water soluble adhesive polymer will dissolve faster than the sheets so that the sheets are released from the laminate structure and one another so that they can be eliminated from the body.

The present invention is not limited to the use of any particular biocompatible material to form the wall of the tubular body. In some embodiments, the biocompatible material is selected from the group consisting of (Poly(α-esters), Polyglycolide, Polylactide, Poly(L-lactic acid) (PLLA), Poly(D-lactic acid) (PDLA), Poly(D,L-lactic acid) (PDLLA), Poly(lactide-co-glycolide), Polyhydroxyalkanoates, Poly(3-hydroxybutyrate), Poly(3-hydroxybutyrate-co-3-hydroxyvalerate (PHBV), Polycaprolactone (PCL), Poly(propylene fumarate) (PPF), Polyanhydrides, Polyacetals, Poly(ortho esters), Polycarbonates, Poly(trimethylene carbonate) (PTMC), Poly(desaminotyrosyltyrosine alkyl ester carbonates) (PDTEs), Polyurethanes, Polyphosphazenes, (Poly[bis(trifluoroethoxy)phosphazene], Polyphosphoesters, Poly(ester ether)s, Polydioxanone (PDO), Poly(β-amino esters) (PBAEs), Poly(anhydride ester)s, Poly(ester urethane)s, Poly(ethylene glycol) (PEG), Poly(propylene glycol) (PPG), triblock pluronic ([PEG]n-[PPG]m-[PEG]n), pluronic, PEG diacrylate (PEGDA), PEG dimethacrylate (PEGDMA), collagen, (Collagen types I, II, III, and IV), elastin and elastin-like polypeptides (ELPs), albumin, fibrin, natural poly(amino acids), poly(γ-glutamic acid), poly(L-lysine), synthetic poly(amino acids), poly(L-glutamic acid), poly(aspartic acid), poly(aspartic acid) (PAA), polysaccharides, hyaluronic acid (HA), chondroitin sulfate (CS), chitin, chitosan, alginate, dextran, agarose, mannan and inulin and combinations thereof.

The present invention is not limited to the use of any particular water soluble adhesive polymer. In some embodiments, the water soluble adhesive polymer is selected from the group consisting of Polyvinyl alcohol, Poly(ethylene glycol), Polyvinyl pyrrolidon, Polyacrylic acid (PAA), Polyacrylamides, N-(2-Hydroxypropyl) methacrylamide (HPMA), Polyoxazoline, Polyphosphates, Polyphosphazenes, xanthan gum, pectins, carrageenan, Cellulose ethers, Carboxymethyl cellulose (CMC), (Hydroxypropyl Cellulose (HPC), Hydroxypropylmethyl Cellulose (HPMC), hyaluronic acid (HA), albumin, starch, starch-based derivatives, and combinations thereof.

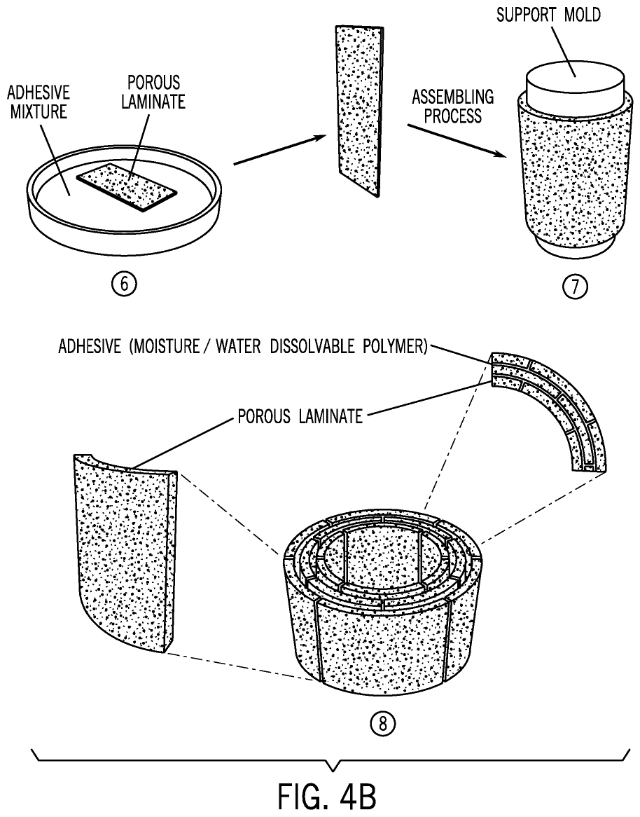

Referring to FIG. 4, the polymer laminates can preferably be made by mixing one or more biocompatible polymers, using suitable solvent (first solvent) to form medium 1 (FIG. 4 (1)). In some embodiments, porosity can be induced to the polymer laminate structure by mixing medium 1 with medium 2, (FIG. 4 (2)), wherein medium 2 can preferably include one or more porosity agents selected from sodium chloride crystals, sugar crystals, baking soda crystals, powders, polymers, hydrogels, and gels that have controllable degradation rates in specific solvents. In some embodiments, the ratio of the porosity agent(s) to the polymer structure can vary from 0.1 to 99.999 wt. %. In some embodiments, the mixture of medium 1 and medium 2 is introduced into a suitable mold (FIG. 4 (3)). In some embodiments, a second solvent is used to remove medium 2 (FIG. 4 (4)), wherein the biocompatible polymer used to form the sheet is insoluble in the second solvent. In some embodiments, the second solvent is selected from water, ethanol, methanol, etc. In some embodiments, the individual polymer laminate dimensions are selected based on the desired properties (FIG. 4 (5)). In some embodiments, the ratio between the biocompatible polymers and the medium 2 is in a range of about 0%-99.999% by weight.

In some embodiments, the polymer laminates comprising medium 1 or a mixture of medium 1 and 2 are deposited onto a suitable substrate or mold by using a deposition device. Suitable deposition devices include, but are not limited to, an injection device, a spraying device such as an air spraying device or an electrospraying device, a thermal spraying device, or a 3D printer. The present invention is not limited to the use of any particular biocompatible polymers or the use of particular porosity agents. In some embodiments, medium 1 may have the following formula(s): a solution of Polyurethanes in ethanol, a solution of chitosan in ethanol, and a blend of Polycaprolactone (PCL) and Polyurethanes in chloroform. In some embodiments, medium 2 may comprise sodium chloride crystals, sugar crystals, or baking soda crystals.

In some embodiments, the polymer laminates are assembled by welding, attachment, adhesion or adherence to form the hollow structure (See, FIG. 4 (6-7)), by using fast moisture/water degradable polymer as described above. The present invention is not limited to the use of any particular water soluble adhesive polymer. As described above, in preferred embodiments, a water soluble adhesive polymer is selected that has a dissolution rate that is faster than the dissolution rate of the biocompatible polymer sheets. Non limiting examples of adhesive polymer formulations include Polyvinyl alcohol, Poly(ethylene glycol), Polyvinylpyrrolidon, and a mixture of Poly(ethylene glycol) and Polyvinylpyrrolidone.

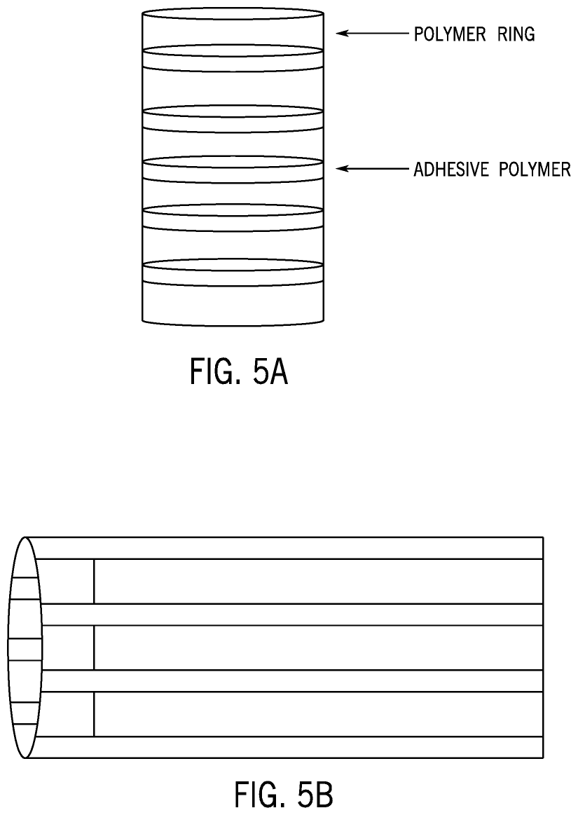

FIGS. 5A and 5B provide an overview of alternative arrangements of the biocompatible polymer sheets to form a tubular body. As shown in FIGS. 5A and 5B, the sheets may be in the form of a series of rings joined by adhesive polymer or arranged longitudinally along the axis of the tubular body and joined by adhesive polymer.

The present invention is not limited to any particular method for making the anastomotic guides. In further embodiments, the tubular bodies of the anastomotic guides of the present invention may be fabricated by printing a moisture and/or water degradable polymer such as PVP to have desired dimension. In some embodiments, pores can optionally be introduced during the printing process. In still other embodiments, the tubular bodies of the anastomotic guides can optionally be fabricated by assembling polymer laminates in multiple rows, e.g., from 1 to 1000 rows. In these embodiments, it is contemplated that each individual laminate can optionally have a different moisture and/or water expansion response so that the devise integrity is based on the moisture/water expansion response of each individual laminate. In preferred embodiments, the device could lose its mechanical stability or completely degrade after a predetermined time. As above, porosity agents can optionally be included and removed after each step or when the device is completely assembled. In still other embodiments, the tubular body can optionally be fabricated by using a multi nozzle bio-printer such that each polymeric composite is deposited in a pre-determined pattern. In still further embodiments, two different polymers (e.g., with fast and slow degradation rates) can optionally be mixed in ratios ranging from 0.01 to 99.99 wt % and printed together in a cylindrical shape. In other embodiments, the two polymers are optionally printed such that the ratio between the laminates and adhesive media vary in the various rows such that the total degradation time will vary as a function of the desired medical outcome and the particular biological environment that the device will be placed into.

Any portion of an anastomotic guide as described herein, for example the biocompatible polymer sheets or the water soluble adhesive polymer, can comprise a therapeutic agent, for example, be coated or imbibed with a therapeutic agent, whether dry, gel or liquid. Examples of therapeutic agents comprise antimicrobial compounds including antimicrobial polypeptides such as defensins and cathelicidin, loracarbef, cephalexin, cefadroxil, cefixime, ceftibuten, cefprozil, cefpodoxime, cephradine, cefuroxime, cefaclor, neomycin/polymyxin/bacitracin, dicloxacillin, nitrofurantoin, nitrofurantoin macrocrystal, nitrofurantoin/nitrofuran mac, dirithromycin, gemifloxacin, ampicillin, gatifloxacin, penicillin V potassium, ciprofloxacin, enoxacin, amoxicillin, amoxicillin/clavulanate potassium, clarithromycin, levofloxacin, moxifloxacin, azithromycin, sparfloxacin, cefdinir, ofloxacin, trovafloxacin, lomefloxacin, methenamine, erythromycin, norfloxacin, clindamycin/benzoyl peroxide, quinupristin/dalfopristin, doxycycline, amikacin sulfate, vancomycin, kanamycin, netilmicin, streptomycin, tobramycin sulfate, gentamicin sulfate, tetracyclines, framycetin, minocycline, nalidixic acid, demeclocycline, trimethoprim, miconazole, colistimethate, piperacillin sodium/tazobactam sodium, paromomycin, colistin/neomycin/hydrocortisone, amebicides, sulfisoxazole, pentamidine, sulfadiazine, clindamycin phosphate, metronidazole, oxacillin sodium, nafcillin sodium, vancomycin hydrochloride, clindamycin, cefotaxime sodium, co-trimoxazole, ticarcillin disodium, piperacillin sodium, ticarcillin disodium/clavulanate potassium, neomycin, daptomycin, cefazolin sodium, cefoxitin sodium, ceftizoxime sodium, penicillin G potassium and sodium, ceftriaxone sodium, ceftazidime, imipenem/cilastatin sodium, aztreonam, cinoxacin, erythromycin/sulfisoxazole, cefotetan disodium, ampicillin sodium/sulbactam sodium, cefoperazone sodium, cefamandole nafate, gentamicin, sulfisoxazole/phenazopyridine, tobramycin, lincomycin, neomycin/polymyxin B/gramicidin, clindamycin hydrochloride, lansoprazole/clarithromycin/amoxicillin, alatrofloxacin, linezolid, bismuth sub salicylate/metronidazole/tetracycline, erythromycin/benzoyl peroxide, mupirocin, fosfomycin, pentamidine isethionate, imipenem/cilastatin, troleandomycin, gatifloxacin, chloramphenicol, cycloserine, neomycin/polymyxin B/hydrocortisone, ertapenem, meropenem, cephalosporins, fluconazole, cefepime, sulfamethoxazole, sulfamethoxazole/trimethoprim, neomycin/polymyxin B, penicillins, rifampin/isoniazid, erythromycin estolate, erythromycin ethylsuccinate, erythromycin stearate, ampicillin trihydrate, ampicillin/probenecid, sulfasalazine, sulfanilamide, sodium sulfacetamide, dapsone, doxycycline hyclate, trimenthoprim/sulfa, methenamine mandelate, plasmodicides, pyrimethamine, hydroxychloroquine, chloroquine phosphate, trichomonocides, anthelmintics, atovaquone, bacitracin, bacitracin/polymyxin b, gentamycin, neomycin/polymyxin/dexameth, neomycin sulf/dexameth, sulfacetamide/prednisolone, sulfacetamide/phenylephrine, tobramycin sulfate/dexameth, bismuth tribromophenate, silver ion compounds, silver nanoparticles, zerovalent silver, multivalent silver, elemental silver, and silver containing compounds such as silver sulfadiazine and related compounds. In some embodiments, the therapeutic agent is a local anaesthetic, for example, bupivacaine, lidocaine, articaine, prilocaine, and mepivacaine. In some embodiments, the therapeutic agent is an opioid, for example, codeine, fentanyl, hydrocodone, hydrocodone and acetaminophen, hydromorphone, meperidine, morphine, oxycodone, oxycodone and acetaminophen, oxycodone and naloxone. In some embodiments, the therapeutic agent is an anti-inflammatory, e.g., adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalac), heteroaryl acetic acids (tolmetin, diclofenac, and ketorolac), and arylpropionic acids (ibuprofen and derivatives). In some embodiments, the therapeutic agent is an angiogenic agents, e.g., vascular endothelial growth factor (VEGF), fibroblast growth factor (FGF) platelet derived growth factor (PDGF), or erythropoietin.

### EXAMPLES

### Example 1

An anastomotic guide of the present invention was used in a porcine model that is relevant to multiple mammalian species. The anastomotic guide for use in the small intestine was developed using ex vivo specimens followed by an in vivo feasibility study to assess the surgeon's ability to use the intraluminal guide during intestinal anastomosis. Anastomoses in the ex vivo study were performed on small intestinal tracts harvested from swine cadavers. These intestinal segments were used to perform hand-sewn, end-to-end anastomosis, with or without the use of a prototype intraluminal guide. Time of completion, burst pressure, and intestinal diameter were assessed. Then, a rapidly degradable intraluminal guide composed of layers of polyurethane and polyvinylpyrrolidone in a hollow cylinder were fabricated to the size of the anticipated bowel lumen in young pigs. The in vivo study was done using 6 pigs in which 2 complete intestinal trans-sections were performed on the small intestine. Of these 2 transectional enterotomies, one was repaired solely with a hand-sewn end-to-end anastomosis, and one repaired with the use of the intraluminal guide and an identical suturing technique. The pigs were monitored for 13 days after which time they were sacrificed and necropsy examinations performed. Burst pressure, maximum luminal diameter, and presence of adhesions were assessed.

### Materials and Methods

### Ex Vivo Investigation

Freshly harvested small intestinal segments from swine cadavers were cut along the mesentery and maintained in cooled saline or water until immediately prior to testing. Segments were trimmed to approximately 20-cm long segments and the intestinal lumens were evacuated and rinsed. Each segment was transected and the halves laid end-to-end so that the cut edges were aligned. Anastomoses were performed using #3-0 PDS suture placed in one of two techniques: 1) simple continuous suturing using two suture segments (each segment hemicircumferential) or 2) simple continuous suturing using two suture segments (each segment hemicircumferential) with the addition of an anastomotic guide (AG) placed prior to the performance of the anastomosis.

For the model of an intraluminal guide, a segment of 0.5-inch diameter PVC pipe was used to mimic the function of an anastomotic guide. This was placed into the lumen of each half of the intestinal segment and the cut edges were aligned. For each trial, a timer was set just before the first suture was placed and stopped immediately after the last knot was tied. Other than the presence of an intraluminal guide, the technique for both of the groups utilizing two suture segments was identical, where suturing began at the mesenteric side and was continued with a simple continuous pattern 180° around to the anti-mesenteric side. This procedure was repeated on the remaining cut edge on the opposite side. For the group utilizing an intraluminal guide and a single suture segment, the technique involved a simple continuous pattern placed 360° around the bowel edges. Regardless of the technique, if any obvious gap was noticed, a single interrupted suture was placed.

Time for completion or the EEA was measured for the performance of each anastomosis, with the timer being set just before the first suture was placed and stopped immediately after the last knot was tied.

Burst pressure was measured by instilling saline into the anastomotic region and observing the maximum pressure withstood by the anastomosis via an arterial pressure monitor. Burst pressure withstood by the anastomotic sites was assessed using a digital pressure monitor. Intraluminal guides were removed from segments in which they were employed and the open ends of each segment were clamped closed, leaving an approximately 12-cm region centered on the anastomosis. A needle was inserted into one side of this region and connected to a bag of saline, and a needle placed into the opposing side attached to the pressure monitor. The lumen was gradually distended with saline while the anastomosis was observed for leaks. Once a leak occurred, the pressure reading was recorded and considered the maximum burst pressure withstood by the anastomotic site for that specimen.

Diameter difference was calculated based on diameter measurements of the intestinal regions proximally and distally adjacent to the anastomosis, as well as at the anastomotic site, while saline remained infused in the segments following burst pressure measurement. While each segment was still filled with fluid, the diameter was measured at six locations: three being anti-mesentery to mesentery axes (proximal to anastomosis, at anastomosis, and distal to anastomosis), and three side-to-side axes (proximal to anastomosis, at anastomosis, and distal to anastomosis). From these diameter measurements, the diameter difference (%) between the proximal and distal regions versus the anastomotic site was determined.

### Intraluminal Guide Fabrication

3D printed models of an intraluminal guide were fabricated based on expected bowel size in an approximately 70 kg pig, as well as length predicted to be of greatest benefit to the efficiency of the performance of an anastomosis. A hollow cylindrical tube was determined to be the ideal shape. These prototypes were used as models for creation of a rapidly degradable, intraluminal anastomotic guide. The desired specifications were that the guide would degrade not less than 30 minutes and not longer than 3 hours after implantation in the intestine.

### In Vivo Investigation

Six domestic cross-bred pigs, weighing 35 to 70 kg, were housed in separate adjacent pens and acclimated to their environment for twelve days. Each pig was fasted for a minimum of 12 hours prior to surgery, and water access was restricted a minimum of 2 hours before surgery. Peri-operative analgesia was provided by placement of transdermal fentanyl patches (1 μg/kg) applied to the back along the dorsal midline in the mid-thoracic region at least 12 hours prior to surgery. Subjects were pre-medicated with xylazine, induced with a combination of midazolam and ketamine, intubated, and maintained under anesthesia on isoflurane. Each subject was placed into dorsal recumbency, clipped and aseptically prepared for ventral midline laparotomy.

The surgical model consisted of a 10-cm ventral midline laparotomy with subsequent exteriorization of 20-40 cm of jejunum. Bowel was milked free of contents and a 15-cm segment isolated with Doyen intestinal clamps. A transverse enterotomy was performed and single interrupted sutures of #3-0 PDS placed at the mesenteric and anti-mesenteric margins of the cut ends for stabilization and to aide in apposition of the cut edges. The anastomosis was completed with an interrupted simple continuous appositional pattern with #3-0 PDS (two suture segments, each placed hemi-circumferentially). Integrity, blood perfusion, and complete closure of the anastomosis was evaluated. Approximately 20-cm distal to the first anastomosis, a second enterotomy was performed in like manner, except after the first single interrupted suture was placed and before closing the cut edges of the bowel with the same technique, a biodegradable intraluminal guide was placed within the lumen traversing and centered on the cut edges. Upon replacement of the jejunum within the abdominal cavity, the linea alba was closed using #0 PDS, the subcutaneous layer with #2-0 PDS, and finally the skin closed with #1 polypropylene, all utilizing a simple continuous pattern.

Pigs were monitored frequently for signs of pain, incision site abnormalities, vomiting, abdominal distention, diarrhea, or constipation. Analgesia was maintained with fentanyl patches (1 ug/kg, TD) for 72 hours peri-operatively, and meloxicam (0.4 mg/kg, PO) once daily for five days. Days 8-13 consisted of visual monitoring twice daily.

All pigs were sacrificed 14 days after surgery and necropsy examinations performed to assess the gross appearance of the bowel and anastomoses and surrounding abdominal cavity.

Maximum bowel diameter was determined for each anastomotic site. Burst pressures were performed in the same manner as for the ex vivo investigation.

### Results

### Ex Vivo Investigation

Surgical procedure time for completion of the EEA procedure, burst pressure achieved at the anastomotic site, maximum diameter of the anastomotic site, intestine immediately proximal and distal to the EEA are summarized in Table 1.

Procedure time required to perform an EEA (without an AG) was a mean of 4 minutes and 14 seconds+/−SD (39%) longer than with the use of an AG.

Burst pressure was similar for each treatment group groups. The maximum diameter % difference at the EEA site as compared with the adjacent proximal and distal intestinal regions was significantly less when an AG was used (Table 1). Specifically, there was between 14.7 and 15.2% less stricture at guide-facilitated anastomoses compared to anastomoses performed without a guide. Lastly, subjective data from those performing the anastomoses revealed that the procedure was easier to perform when there was a guide within the lumen.

TABLE 1
Comparison of the average time for completion, burst pressure,
and diameter difference for each anastomotic technique.

| | 2 Suture | 2 Suture Segments, |
|---|---|---|
| | Segments, | Anastomotic |
| | Hand-sewn EEA | Guide EEA |
| Number of Trials | 10 | 9 |
| Time for Completion (min:sec) | 15:04 | 10:50 |
| Burst Pressure (mmHg) | 48 | 43 |
| Diameter Difference (%) | 78 | 93 |

### In Vivo Investigation

During the first few days post-operatively, there were no complications other than a few occasions of diarrhea in some of the subjects and fluctuating low-grade fevers that resolved with antibiotic treatment. During the latter end of the recovery period, mild swelling was noted at the incision site of a few of the pigs.

Following sacrifice of the pigs, necropsies were performed during which gross examination of the anastomoses and surrounding abdominal cavity was performed. Adhesions were discovered at EEA sites and some adjacent regions within the abdominal cavity, but there was no significant difference between the anastomotic sites that involved or did not involve the use of the AG One EEA in 1 pig was noted to have had minor dehiscence at the EEA site of the hand-sewn anastomosis; no leakage or dehiscence were noted in any of the EEA done with the AG. The gross appearance of the healed margins of the bowel were similar for all EEA sites.

Burst pressure was found to be approximately 10% greater at anastomotic sites that were facilitated using an AG when compared to hand-sewn EEA sites (Table 2). This difference was not statistically significant (Table 2). The maximum diameter achieved at the anastomosis site that utilized an AG was significantly greater than that achieved using the hand-sewn anastomoses (Table 2). Subjective evaluation by surgeons performing the anastomoses noted that the guide aided in the placement of more evenly spaced suture bites and eased the performance of the EEA. The surgeons noted that there was some difficulty placing the guide within the lumen due to its pliability.

TABLE 2
Comparison of the average number of adhesions at the anastomotic site,
burst pressure, and maximum diameter for each anastomotic technique.

| | Anastomosis with No | Anastomosis |
|---|---|---|
| | Guide | Facilitated by Guide |
| Number of Adhesions at Site | 1 | 1 |
| Burst Pressure (mmHg) | 150.6 | 166.0 |
| Maximum Diameter (mm) | 22.7 | 26.6 |
| Diameter difference | | +17% |

Presence of adhesions at the anastomotic sites and local regions of the abdominal cavity was assessed grossly. Burst pressure was measured by instilling saline into the anastomotic region and observing the maximum pressure withstood by the anastomosis via an arterial pressure monitor. Maximum diameter at each anastomotic site was measured while saline remained infused in the segments following burst pressure measurement.

### Discussion

The ex vivo investigation revealed that the use of and anastomotic guide reduces the surgical procedure time for completion of an end-to-end anastomosis. This is likely due to the ability to place sutures more easily within the cut edges of bowel due to the edges being dilated by the guide as opposed to the natural contraction and eversion that occurs when the bowel is transected. Precision and accuracy in reconstruction of the continuity and patency of the bowel is critical to ensuring that devastating dehiscence or obstruction associated with structure does not occur.

In the ex vivo investigation, intestinal segment diameter was maximized when using an AG to facilitate the EEA, suggesting that patients in which this device is used might have a reduce risk of leakage, dehiscence, and stricture. In the in vivo investigation, anastomotic site diameter was improved in the sites in which an AG was used. Although small, this difference may be clinically significant resulting in a decreased likelihood of stricture and impaction at surgical sites.

Burst pressures measured between the groups in both the ex vivo and in vivo investigations were not significantly different. This suggests that the healing process in the intestine with EEA is similar regardless of technique used. Burst pressures achieved in the in vivo experiment were physiologically appropriate, so it does not appear that the performance of anastomoses produced a risk of leakage, at least when assessed two weeks post-operatively.

Adhesion development in the in vivo investigation was noted to occur at nearly all anastomotic sites and within local areas of the abdominal cavity. It was difficult to differentiate which anastomotic site may have incited the additional adhesions within the abdomen. Intraluminal appearance of each anastomosis was not noticeably different supporting the likelihood that the methods did not adversely affect the normal process of intestinal healing.

The EEA anastomotic technique was noted by the surgeons to be easier to perform with the use of a guide in both the ex vivo and in vivo trials. The only concern noted with the use of the AG in the in vivo investigation regarded difficulty when placing the guide within the lumen due to its pliability. This may be addressed in modified designs by alterations in thickness or polymer composition. The degradation time of the guide was assessed in hydration studies prior to placement within the subjects and was deemed appropriate. No remnants remained within the lumen upon necropsy evaluation, which further supports that the guides indeed degraded.

One concern about placement of a medical device within the bowel lumen is the potential for dislodgement, migration, interruption of motility, and obstruction. We designed a rapidly degradable polymeric device to avoid these potential complications. Should the guide dislodge shortly after the surgery, it would quickly degrade with the passage of digesta within the lumen.

The ability of an intraluminal anastomotic guide to aid in increasing the diameter of an intestinal anastomosis site, as well as ease the performance of the technique itself, without presenting any additional complications, supports the use of guides for this particular procedure. This could ultimately reduce complications that occur post-operatively, including dehiscence, leakage, peritonitis, stricture, and impaction. Any reduction in time of performance would also be beneficial as some patients undergoing this procedure may be physiologically and anesthetically unstable. The use of a swine model is advantageous for translation to human medicine, as swine have gastrointestinal tracts that are very similar to humans.

### REFERENCES

[1] Kate, Vikram, MBBS, M S, PhD, FACS, FACG, FRCS(Edin), FRCS(Glasg), FIMSA, MAMS, MASCRS, Raja Kalayarasan, MBBS, M S, MCh, Anup Mohta, MBBS, M S, MCh, and Kurt E. Roberts, M D. “Intestinal Anastomosis.” Medscape. WebMD LLC, 11 Mar. 2016. Web. 30 Jun. 2017. <http://emedicine.medscape.com/article/1892319-overview>.
[2] Kate, Vikram, MBBS, M S, PhD, FACS, FACG, FRCS(Edin), FRCS(Glasg), FIMSA, MAMS, MASCRS, Raja Kalayarasan, MBBS, M S, MCh, Anup Mohta, MBBS, M S, MCh, and Kurt E. Roberts, M D. “Intestinal Anastomosis Technique.” Medscape. WebMD LLC, 11 Mar. 2016. Web. 30 Jun. 2017. <http://emedicine.medscape.com/article/1892319-technique >.
[3] Goulder, Frances. “Bowel Anastomoses: The Theory, the Practice and the Evidence Base.” World Journal of Gastrointestinal Surgery 4.9 (2012): 208-13. Web. 30 Jun. 2017.
[4] Fielding L P, Stewart-Brown S, Blesovsky L, Kearney G. “Anastomotic integrity after operation for large-bowel cancer: a multicentre study.” Br Med J 281 (1980): 411-14.
[5] Deveney K E, Way L W. “Effect of different absorbable sutures on healing of gastrointestinal anastomoses.” Am J Surg 133.1 (1977): 86-94.
[6] Burch J M, Franciose R J, Moore E E, Biffi W L, Offner P J. “Single-layer continuous versus two-layer interruptes intestinal anastomosis: a prospective randomized trial.” Ann Surg 231.6 (2000): 832-7.
[7] Shikata S, Yamagishi H, Taji Y, Shimada T, Noguchi Y. “Single-versus two-layer intestinal anastomosis: a meta-analysis of randomized controlled trials.” BMC Surg 6.2 (2006).
[8] Garude K, Tandel C, Rao S, Shah N J. “Single layered intestinal anastomosis: a safe and economic technique.” Indian J Surg 75.4 (2013): 290-3.
[9] Sajid M S, Siddiqui M R, Baig M K. “Single layer versus double layer suture anastomosis of the gastrointestinal tract.” Cochrane Database Syst Rev (2012): 1:CD005477.
[10] Shandall A, Lowndes R, Young H L. “Colonic anastomotic healing and oxygen tension.” Br J Surg 72.8 (1985): 606-9.
[11] Getzen L C, Roe R D, Holloway C K. “Comparative study of intestinal anastomotic healing in inverted and everted closures.” Surg Gynecol Obstet 123.6 (1966): 1219-27.
[12] Goligher J C, Morris C, McAdam W A. De Dombal F T, Johnston D. “A controlled trial of inverting versus everting intestinal suture in clinical large-bowel surgery.” Br J Surg 57.11 (1970): 817-22.
[13] Lustosa S A, Matos D, Atallah A N, Castro A A. “Stapled versus handsewn methods for colorectal anastomosis surgery.” Cochrane Database Syst Rev (2001): CD003144.
[14] Scher K S, Scott-Conner C, Jones C W, Leach M. “A comparison of stapled and sutured anastomoses in colonic operations.” Surg Gynecol Obstet 155 (1982): 489-493.
[15] Didolkar M S, Reed W P, Elias E G, Schnaper L A, Brown S D, Uhaudhary S M. “A prospective randomized study of sutured versus stapled bowel anastomoses in patients with cancer.” Cancer 57 (1986): 456-60.
[16] Chassin J L, Rifkind K M, Sussman B, Kassel B, Fingaret A, Drager S, Chassin P S. “The stapled gastrointestinal tract anastomosis: incidence of postoperative complications compared with the sutured anastomosis.” Ann Surg 188 (1978): 689-96.
[17] Reiling R B, Reiling W A, Bernie W A. Huffer A B, Perkins N C, Elliott D W. “Prospective controlled study of gastrointestinal stapled anastomosis.” Am J Surg 139 (1980): 147-52.
[18] West of Scotland and Highland Anastomosis Study Group. “Suturing or stapling in gastrointestinal surgery: a prospective randomized study.” Br J Surg 78 (1991): 337-41.
[19] MacRae H M, McLeod R S. “Handsewn vs. stapled anastomoses in colon and rectal surgery: a meta-analysis.” Dis Colon Rectum 41.2 (1998): 180-9.
[20] Choy P Y, Bissett I P, Docherty J G, Parry B R, Merrie A E. “Stapled versus handsewn methods for ileocolic anastomoses.” Cochrane Database Syst Rev 3 (2007): CD004320.
[21] Brundage S I, Jurkovich G J, Grossman D C, Tong W C, Mack C D, Maier R V. “Stapled versus sutured anastomoses in the trauma patient.” J Trauma 47 (1999): 500-7. Web. 30 Jun. 2017.
[22] Witzke J D, Kraatz J J, Morken J M, Ney A L, West M A, Van Camp J M, Zera R T, Rodriguez J L. “Stapled versus hand sewn anastomoses in patients with small bowel injury: a changing perspective.” J Trauma 49 (2000): 660-605. Web. 30 Jun. 2017.
[23] Brundage S I, Jurkovich G J, Hoyt D B, Patel N Y, Ross S E, Marburgger R, Stoner M, Ivatury R R, Ku J, Rutherford E J, Maier R V. “Stapled versus sutured gastrointestinal anastomoses in the trauma patient: a multicenter trial.” J Trauma 51 (2001): 1054-61.
[24] Leung T T, MacLEan A R, Buie W D, Dixon E. “Comparison of stapled versus handsewn loop ileostomy closure: a meta-analysis.” J Gastrointest Surg 12 (2008): 939-44.
[25] Saha A K, Tapping C R, Foley G T, Baker R P, Sagar P M, Burke D A, Sue-Ling H M, Finan P J. “Morbitity and mortality after closure of loop ileostomy.” Colorectal Dis 11 (2009): 866-71.
[26] Naumann, David N., MRCS, Aneel Bhangu, PhD, Michael Kelly, MBChB, and Douglas M. Bowley, FRCS. “Stapled versus Handsewn Intestinal Anastomosis in Emergency Laparotomy: A Systemic Review and Meta-analysis.” Surgery 157.4 (2015): 609-18. Web. 30 June 2017.
[27] Farrah J P, Lauer C W, Bray M S, McCartt J M, Change M U, Meredith J W, et al. “Stapled versus hand-sewn anastomoses in emergency general surgery: a retrospective review of outcomes in a unique patient population.” J Trauma Acute Care Surg 74.1 (2013): 187-92.
[28] Catena F, La Donna M, Gagliardi S, Avanzolini A, Taaffurelli M. “Stapled versus hand-sewn anastomoses in emergency intestinal surgery: results of a prospective randomized study.” Surg Today 34 (2004):123-6. Web. 30 Jun. 2017.
[29] Tobias, K M, Ayres, R. Key Gastrointestinal Surgeries: Intestinal Anastomosis. Proc. of Veterinary Medicine: C E Symposium. 4th ed. Vol. 101. 2006. 226-9. Advanstar Communications, Inc. Web. 3 Jul. 2017. <http://go.galegroup.com/ps/i.do?&id=GALE|A145690345&v=2.1&u=tel_a_utl&it=r&p=AONE&sw=w&authCount=1>.
[30] Ralphs S C, Jessen C R, Lipowitz A J. “Risk factors for leakage following intestinal anastomosis in dogs and cats: 115 cases (1991-2000).” J Am Vet Med Assoc 223.1 (2003):665-82.
[31] Weisman D L, Smeak D D, Birchard S J, et al. “Comparison of a continuous suture pattern with a simple interrupted pattern for enteric closure in dogs and cats: 83 cases (1991-1997).” J Am Vet Med Assoc 214.10 (1999): 1507-10. Web. 3 Jul. 2017.
[32] Brown, C D. “Small intestines.” Textbook of small animal surgery. By Douglas H. Slatter. 3rd ed. Philadelphia, Pa.: Saunders, 2003. 644-64.
[33] Milovancev M, Weisman D L, Palmisano M P. “Foreign body attachment to polypropylene suture material extruded into the small intestine lumen after enteric closure in three dogs.” J Am Vet Med Assoc 225.11 (1982): 265-8. Web. 3 Jul. 2017.
[34] Holt, D E. “Large intestines.” Textbook of small animal surgery. By Douglas H. Slatter. 3rd ed. Philadelphia, Pa.: Saunders, 2003. 665-82.
[35] Bellenger C R. “Comparison of inverting and appositional methods for anastomosis of the small intestine in cats.” Vet Rec 110.12 (1982): 265-8. Web. 3 Jul. 2017.
[36] Ellison G W, Jokinen M P, Park R D. “End-to-end approximating intestinal anastomosis in the dog: a comparative fluorescein dye, angiographic, and histopathologic evaluation.” J Am Anim Hosp Assoc 18.5 (1982): 729-36. Web. 3 Jul. 2017.
[37] Coolman B R, Ehrhart N, Pijanowski G, et al. “Comparison of skin staples with sutures for anastomosis of the small intestine of dogs.” Vet Surg 29.4 (2000): 293-302. Web. 3 Jul. 2017.
[38] Allen D A, Smeak D D, Schertel E R. “Prevalence of small intestinal dehiscence and associated clinical factors: a retrospective study of 121 dogs.” J An Anim Hosp Assoc 28.1 (1992): 70-6.
[39] Kudisch M, Pavletic M M. “Subtotal colectomy with surgical stapling instruments via a trans-cecal approach for treatment of acquired megacolon in cats.” Vet Surg 22.6 (1993): 457-63. Web. 3 Jul. 2017.
[40] Ullman S L, Pavletic M M, Clark G N. “Open intestinal anastomosis with surgical stapling equipment in 24 dogs and cats.” Vet Surg 20.6 (1991): 385-391. Web. 3 Jul. 2017.
[41] Fossum T W. “Surgery of the digestive system.” Small animal surgery. By Douglas H. Slatter. 2nd ed. St. Louis, Mo.: Mosby, 2002. 369-414.

All publications and patents mentioned in the above specification are herein incorporated by reference. Various modifications and variations of the described devices and methods of the invention will be apparent to those skilled in the art without departing from the scope and spirit of the invention. Although the invention has been described in connection with specific preferred embodiments, it should be understood that the invention as claimed should not be unduly limited to such specific embodiments. It will be evident to those skilled in the art that various modifications may be made, especially in matters of structure, materials, elements, components, shapes, sizes, and arrangements of parts including combinations within the principles described herein, to the full extent indicated by the broad, general meaning of the terms in which the appended claims are expressed. Indeed, various modifications of the described modes for carrying out the invention that are obvious to those skilled in medical devices, the medical and veterinary arts, or related fields are intended to be within the scope of the following claims.

1. An anastomotic guide comprising:
a tubular body comprising a wall having an abluminal surface, a luminal surface and two ends;
the wall comprising at least one sheet of a biocompatible material in a laminate structure, the layers of the laminate structure being joined by a water soluble adhesive polymer, the tubular body being insertable into the lumen of an organ having a luminal surface so that the abluminal surface of the anastomotic guide contacts the luminal surface of the organ.

2. The anastomotic guide of claim 1, wherein the biocompatible material is selected from the group consisting of (Poly(α-esters), Polyglycolide, Polylactide, Poly(L-lactic acid) (PLLA), Poly(D-lactic acid) (PDLA), Poly(D,L-lactic acid) (PDLLA), Poly(lactide-co-glycolide), Polyhydroxyalkanoates, Poly(3-hydroxybutyrate), Poly(3-hydroxybutyrate-co-3-hydroxyvalerate (PHBV), Polycaprolactone (PCL), Poly(propylene fumarate) (PPF), Polyanhydrides, Polyacetals, Poly(ortho esters), Polycarbonates, Poly(trimethylene carbonate) (PTMC), Poly(desaminotyrosyltyrosine alkyl ester carbonates) (PDTEs), Polyurethanes, Polyphosphazenes, (Poly[bis(trifluoroethoxy)phosphazene], Polyphosphoesters, Poly(ester ether)s, Polydioxanone (PDO), Poly(β-amino esters) (PBAEs), Poly(anhydride ester)s, Poly(ester urethane)s, Poly(ethylene glycol) (PEG), Poly(propylene glycol) (PPG), triblock pluronic ([PEG]n-[PPG]m-[PEG]n), pluronic, PEG diacrylate (PEGDA), PEG dimethacrylate (PEGDMA), collagen, (Collagen types I, II, III, and IV), elastin and elastin-like polypeptides (ELPs), albumin, fibrin, natural poly(amino acids), poly(γ-glutamic acid), poly(L-lysine), synthetic poly(amino acids), poly(L-glutamic acid), poly(aspartic acid), poly(aspartic acid) (PAA), polysaccharides, hyaluronic acid (HA), chondroitin sulfate (CS), chitin, chitosan, alginate, dextran, agarose, mannan and inulin and combinations thereof.

3. The anastomotic guide of claim 1, wherein the biocompatible material is a biodegradable material.

4. The anastomotic guide of claim 1, wherein the biodegradable material is water soluble.

5. The anastomotic guide of claim 1, wherein the sheet of biocompatible material is porous.

6. The anastomotic guide of claim 1, wherein the water soluble adhesive polymer is selected from the group consisting of Polyvinyl alcohol, Poly(ethylene glycol), Polyvinyl pyrrolidon, Polyacrylic acid (PAA), Polyacrylamides, N-(2-Hydroxypropyl) methacrylamide (HPMA), Polyoxazoline, Polyphosphates, Polyphosphazenes, xanthan gum, pectins, carrageenan, Cellulose ethers, Carboxymethyl cellulose (CMC), (Hydroxypropyl Cellulose (HPC), Hydroxypropylmethyl Cellulose (HPMC), hyaluronic acid (HA), albumin, starch, starch-based derivatives, and combinations thereof.

7. The anastomotic guide of claim 1, wherein the water soluble adhesive polymer has a dissolution rate in water or aqueous solution that is greater than the dissolution rate of the at least one sheet of biocompatible material.

8. The anastomotic guide of claim 1, wherein the guide further comprises a plurality of sheets of biocompatible material in a laminate structure having two or more layers, the layers of the laminate structure being joined by the water soluble adhesive polymer.

9. The anastomotic guide of claim 1, wherein the tubular body has a diameter compatible with insertion into the small intestine of a mammal so that the abluminal surface of the tubular body engages the luminal surface of the small intestine so that the guide is maintained in place at the site of anastomosis.

10. The anastomotic guide of claim 1, wherein the mammal is selected from the group consisting of humans, non-human primates, pigs, horses, cows, sheep, goats, camelids, dogs and cats.

11. The anastomotic guide of claim 1, further comprising one or more retention members positioned proximal to one or both of the two ends of the tubular body, the retention member(s) providing pressure to the luminal surface of the organ so that when the anastomotic guide is inserted into the organ the position of the anastomotic guide is maintained relative to the luminal surface of the organ.

12. The anastomotic guide of claim 1, wherein the tubular body has a center and has one or more grooves distal to the center of the tubular body that extend around the tubular body so that when the anastomotic guide is inserted into an organ, the grooves are engageable by a retention member external to the organ to maintain the position of the anastomotic guide relative to the luminal surface of the organ.

13. The anastomotic guide of claim 1, wherein one or both of the biocompatible polymer and water soluble adhesive polymer comprise a therapeutic agent.

14. The anastomotic guide of claim 1, wherein the therapeutic agent is an antimicrobial agent.

15. An anastomotic guide comprising:
a tubular body comprising a wall having an abluminal surface, a luminal surface and two ends;
the wall comprising at least one sheet of a porous biodegradable material in a laminate structure having two or more layers, the layers of the laminate structure being joined by a water soluble adhesive polymer so that the tubular body is insertable into the lumen of an organ having a luminal surface so that the abluminal surface of the anastomotic guide contacts the luminal surface of the organ,
wherein the tubular body has a diameter compatible with insertion into the small intestine of a mammal selected from the group consisting of humans, non-human primates, pigs, horses, cows, sheep, goats, camelids, dogs and cats.

16. An anastomosis procedure comprising:
inserting the anastomotic guide of claim 1 into first and second ends of an organ having a lumen so that the anastomotic guide extends between the first and second ends, and
joining the first and second ends by anastomosis.

17. The method of claim 16, wherein the anastomosis comprises joining the first and second ends of the organ by sutures.

18. The method of claim 16, wherein the anastomosis comprises joining the first and second ends of the organ by staples.

19. The method of claim 16, wherein the anastomosis joins ends of an organ resulting from resection of the organ.

20. The method of claim 16, wherein the organ is the small intestine of a mammal.

21. The method of claim 20, wherein the mammal is selected from the group consisting of humans, non-human primates, pigs, horses, cows, sheep, goats, camelids, dogs and cats.

22. The method of claim 16, wherein the anastomotic guide degrades within a period of from 1 minute to 90 days.

23. (canceled)
